(19) United States
(12) Patent Application Publication
(10) Pub. No.: US 2012/0053566 A1
(43) Pub. Date: Mar. 1, 2012

(54) METHOD FOR TREATMENT OF EMPHYSEMA

(75) Inventors: Yuichi TADA, Ashigarakami-gun (JP); Takao Anzai, Ashigarakami-gun (JP); Atsuhiko Nogawa, Ashigarakami-gun (JP)

(73) Assignee: Terumo Kabushiki Kaisha, Shibuya-ku (JP)

(21) Appl. No.: 13/216,949

(22) Filed: Aug. 24, 2011

Related U.S. Application Data
(60) Provisional application No. 61/376,832, filed on Aug. 25, 2010.

(51) Int. Cl. A61M 25/10

(52) U.S. Cl. 604/514

(57) ABSTRACT

Disclosed is a method for treatment of emphysema by which it is possible to reduce the volume of pulmonary alveoli or alveolar sacs abnormally enlarged with destruction by emphysema. The method for treatment of emphysema includes: (a) inserting a catheter having a balloon into a bronchus or bronchiole; (b) dilating the balloon to occlude the bronchus or bronchiole; (c) shrinking pulmonary alveoli or alveolar sacs on the downstream side of the bronchus or bronchiole occluded in the step (b); and (d) injecting a hardening agent through the catheter into the respiratory region and hardening the hardening agent.

### BACKGROUND

1. Technical Field

The present invention relates to a method for treatment of emphysema. More particularly, the invention relates to a method for treatment of emphysema by reducing the volume of the pulmonary alveoli or alveolar sac which have been anomalously expanded with destruction by emphysema.

2. Description of the Related Art

Chronic obstructive pulmonary disease (COPD) means a large group of pulmonary diseases that hinder normal respiration, and is a disease which brings about lung occlusion arising from the presence of at least one selected from asthma, emphysema and chronic bronchitis. COPD frequently involves these symptoms at the same time and makes it difficult to confirm in each case which one of the diseases is actually the cause of the lung occlusion. A case is clinically diagnosed as COPD by constant decrease in expiration flow from the lung over several months, which continues for two years or more in the case of chronic bronchitis. Two of the most critical states relating to COPD are chronic bronchitis and emphysema.

Among these, the emphysema designates a state of anomalous expansion with destruction that occurs in respiratory bronchioles, serving as gas exchange sites, and/or the tissues called alveolar parenchyma such as pulmonary alveoli, alveolar sacs, etc. The alveolar parenchyma in its normal state shrinks at the time of expiration, but the one suffering from emphysema does not recover itself after expansion by respiration. This inhibits satisfactory expiration. Moreover, emphysema decreases the effective area and vascular bed (capillary vessels running point to point on the surface of the pulmonary alveoli) of the pulmonary alveoli, thereby reducing the gas exchange capacity of the lung as a whole. In addition, emphysema involves inflammation that destroys elastin and collagen, thereby causing the lung to decrease elasticity. This result is that the lung cannot keep stretching and expanding the respiratory tract, and this permits easy deformation of the bronchia. Consequently, the bronchus is compressed to become narrow by its surrounding air-filled pulmonary alveoli as the lung shrinks at the time of expiration. This makes the lung expand excessively, preventing air from being discharged easily (see WO 2009/075106 A1, ST MARIANNA UNIVERSITY SCHOOL, MIYAZAWA TERUOMI, “STENT FOR TREATING CHRONIC OBSTRUCTIVE PULMONARY DISEASE”). For this reason, a patient of emphysema purses up his lips to expire (see Jadranka Spahija et al., “Effects of imposed Pursed-Lips Breaching on Respiratory Mechanics and Dyspnea at Rest and During Exercise in COPD”, Chest 2005; 128:640-650).

In Japan, there are above 50,000 patients with emphysema who are receiving home oxygen therapy, and it is estimated that about three million people including those of mild case are liable to emphysema. At present, the major method of therapy for emphysema is home oxygen therapy. Oxygen therapy is often used for the patient who cannot take in sufficient oxygen from air on account of the seriously damaged lung function. However, it merely relieves the patient's condition but is not an effective method of therapy. On the other hand, there are several methods of pharmacotherapy including: administration of bronchodilator to open respiratory tracts in the lung, thereby alleviating breathing difficulties; administration of steroid by inhalation or mouth, thereby alleviating inflammation in respiratory tracts; administration of antibiotics to prevent or treat secondary infection; and administration of expectorant to remove mucus from respiratory tracts (see Jan A. van Noord et al., “Effects of Tiotropium With and Without Formoterol on Airflow Obstruction and Resting Hyperinflation in Patients With COPD”, Chest 2006; 129:509-517). All of these pharmacotherapies help control emphysema and alleviate its symptom, but they are not necessarily effective. In addition, there are several methods of surgical treatment which include lung reductive surgery in which removal of damaged parts from the lung is made to promote expansion of the normal parts of the lung and lung transplantation; however, these surgical methods have disadvantages of a heavy burden on the patient and difficulties in securing the lung for substitution (see Ware J H, et al., “Cost effectiveness of Lung-Volume-Reduction Surgery for Patients with Severe Emphysema”, The New England Journal of Medicine 2003; 348:2092-2102; National Emphysema Treatment Trial Research Group, “A Randomized Trial Comparing Lung-Volume-Reduction Surgery with Medical Therapy for Severe Emphysema”, The New England Journal of Medicine 2003; 348:2059-2073).

If it is possible to carry out “LVR (Lung Volume Reduction)” noninvasively without thoracotomy, many patients can have the chance of therapy. However, the current noninvasive therapy is limited in possibilities of success. For example, one of the noninvasive therapies which are expected to produce the same effect as “LVRS (Lung Volume Reduction Surgery)” is an indwelling device to be left in the bronchus. This device has a one-way valve that prevents the entry of inspired gas into the lung end (see Alferness, Clifton A et al., Spiration, Inc., U.S. Pat. No. 6,258,100 B1). However, once left indwelling in the lung, it prevents access to any place beyond its indwelling point (see Mark L. Mathis, PneumRx, Inc., U.S. Pat. No. 7,549,984 B1). In addition, as another non-surgical means for achieving a reduction in the lung volume, there has been disclosed a method in which a region of the lung is collapsed, a part of the collapsed region is bonded to another region of the lung, and growth of fiber in the bonded tissue or the vicinity thereof is promoted to thereby realize the LVR (see Edward P. Ingenito et al., Bistech, Inc., U.S. Pat. No. 6,682,520 B1). In this method, however, it needs a certain length of time for the lung parenchyma to be destroyed by the reaction of the living body. The U.S. Pat. No. 6,682,520 B1 further describes a method for trying LVR by means of a material containing that part of the damaged lung tissue to which targeting therapy is applied. This method, however, needs the part for targeting therapy and also needs a process in which the material reacts with the damaged part (see Gong; Glen et al., PneumRx, Inc., U.S. Pat. No. 7,678,767 B1).

Thus, at present, no effective method for treatment of emphysema exists in the relevant field of art.

### SUMMARY

It is an object of the present invention to provide a method for reducing the volume of the pulmonary alveoli or alveolar sac suffering from emphysema.

In accordance with one embodiment of the present invention, there is provided a method for treatment of emphysema including: (a) inserting a catheter having a balloon into a bronchus or bronchiole; (b) dilating the balloon to occlude the bronchus or bronchiole; (c) shrinking pulmonary alveoli or alveolar sacs on a downstream side of the bronchus or bronchiole occluded by the step (b); and (d) injecting a hardening agent through the catheter into the respiratory region and hardening the hardening agent.

According to this method pertaining to the present invention, it is possible to less-invasively reduce the volume of pulmonary alveoli or alveolar sacs suffering from emphysema.

### BRIEF DESCRIPTION OF DRAWINGS

FIGS. 1A to 1F are schematic sectional views showing the procedure of steps in a preferred method according to one embodiment of the present invention.

FIGS. 2A to 2G are schematic sectional views showing the procedure of steps a preferred method according to another embodiment of the present invention.

FIGS. 3A and 3B are schematic sectional views showing a preferred embodiment of step (a) in the method pertaining to the present invention.

### DETAILED DESCRIPTION OF THE PREFERRED EMBODIMENTS

The method for treatment of emphysema according to one embodiment of the present invention includes: (a) inserting a catheter having a balloon into a bronchus or bronchiole; (b) dilating the balloon to occlude the bronchus or bronchiole; (c) shrinking pulmonary alveoli or alveolar sacs on a downstream side of the bronchus or bronchiole occluded by the step (b); and (d) injecting a hardening agent through the catheter into the respiratory region and hardening the hardening agent. Incidentally, the term “treatment” as used herein means any medical practice to heal, alleviate, reduce, restore, prevent, or improve emphysema, symptoms of emphysema, or any symptoms that follow emphysema.

According to the present invention, it is made possible to efficiently remove stagnant air in pulmonary alveoli or alveolar sacs (hereinafter referred also to simply as “alveolar parenchyma”) suffering from emphysema. It is also made possible to alleviate or suppress overexpansion of the lung which weakens the patient due to emphysema or occlusion of air-supply bronchi to maintain the reduced volume by respiration. Further, it is made possible to restore the alveolar parenchyma suffering from emphysema to its original size or less, thereby suppressing or preventing compression or occlusion of the surrounding bronchi by the surrounding alveolar parenchyma. In addition, the treatment method according to the embodiment of the present invention resides in therapy by use of a catheter and does not need a surgical treatment, so that the burden on the patient can be alleviated.

Incidentally, alveolar parenchyma suffering from emphysema often has holes, called bypass, that connect the alveolar parenchyma to the surrounding pulmonary alveoli. In relation to this point, the method according to the embodiment of the present invention is particularly effectively applicable to alveolar parenchyma suffering from emphysema wherein no such bypass is present.

Now, the present invention will be described in detail below referring to the drawings.

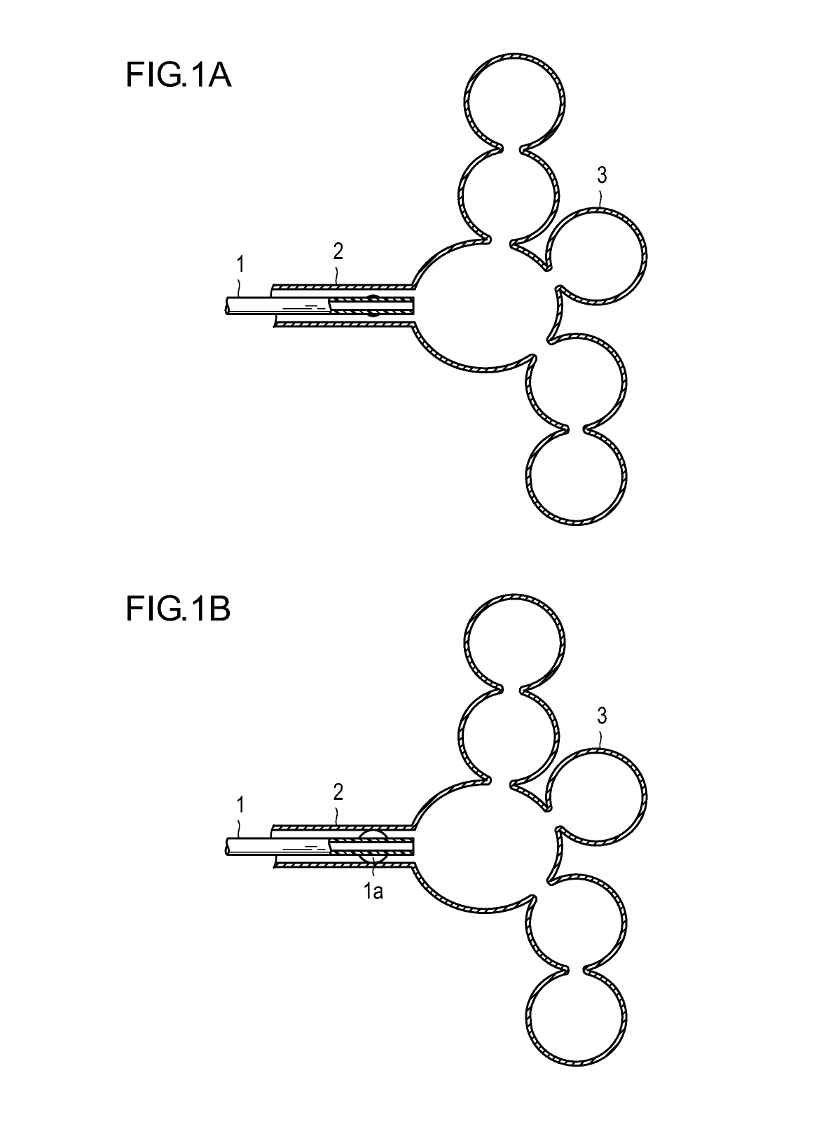

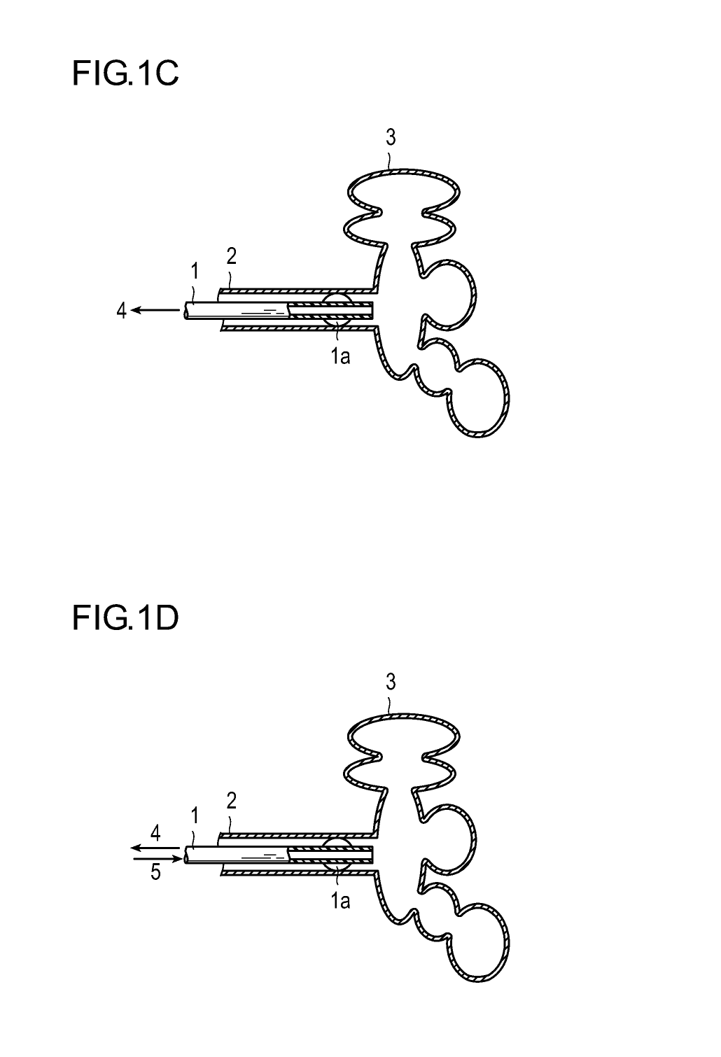

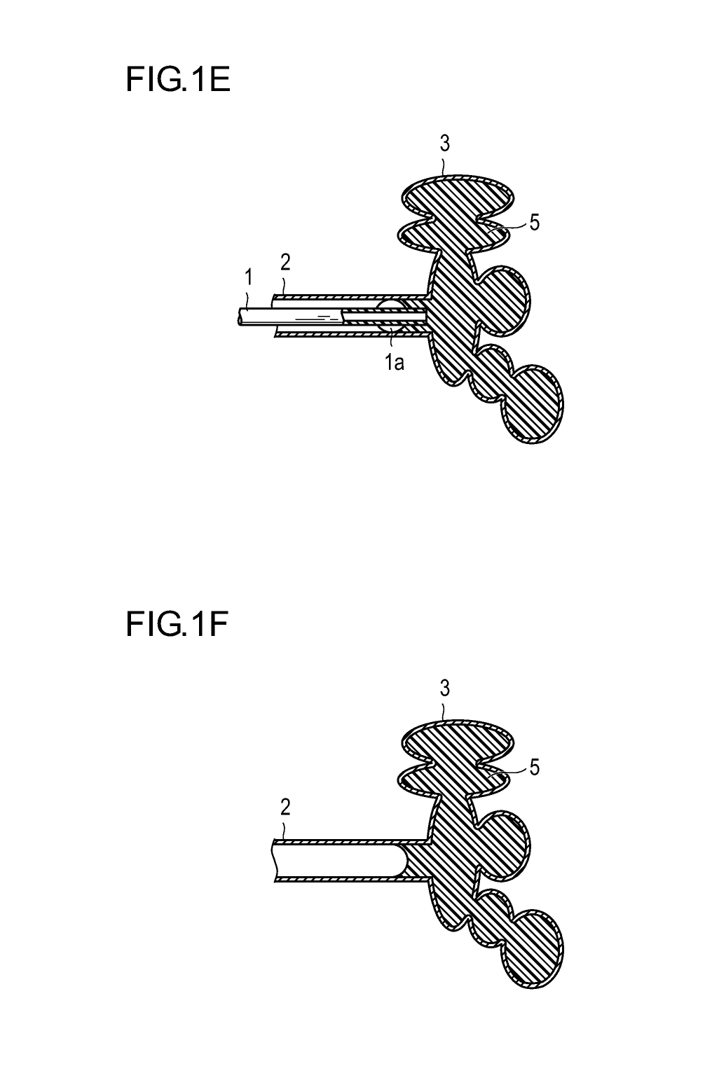

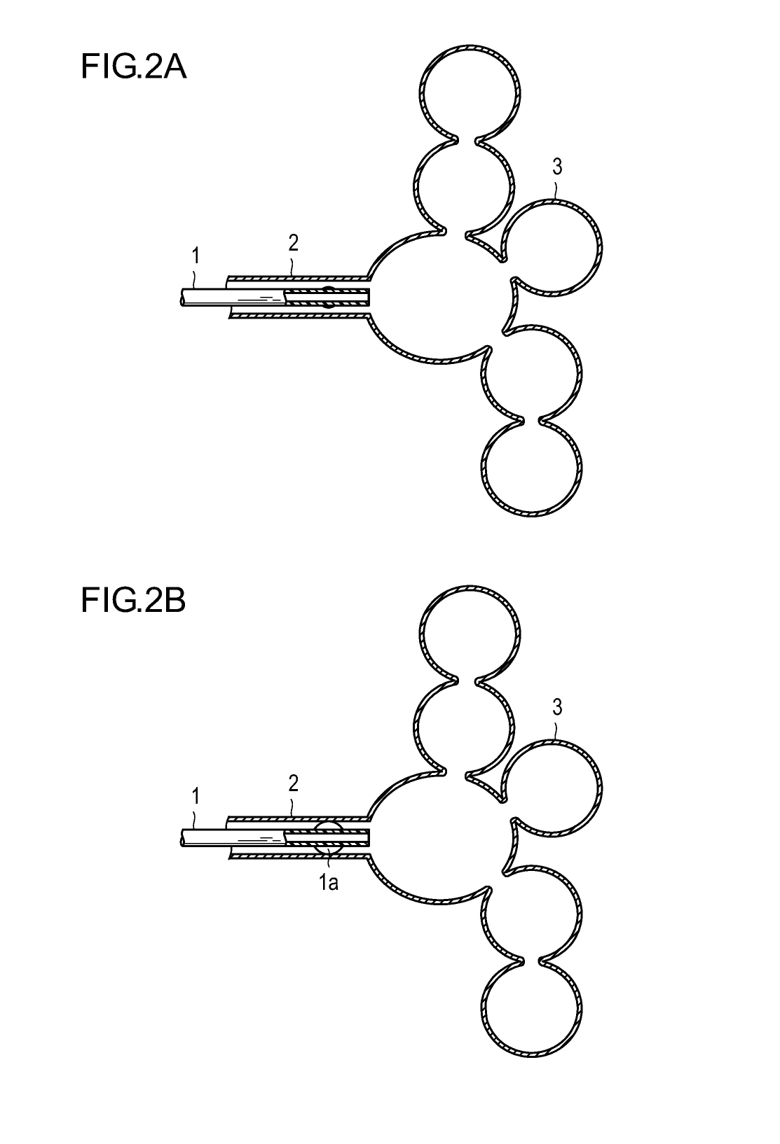

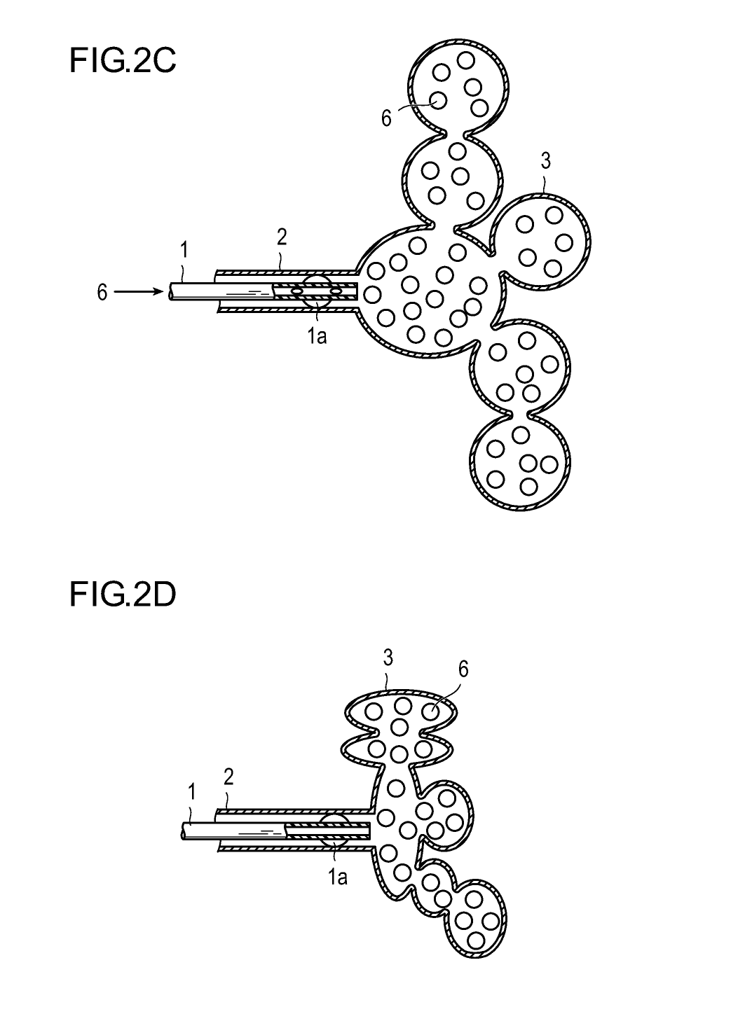

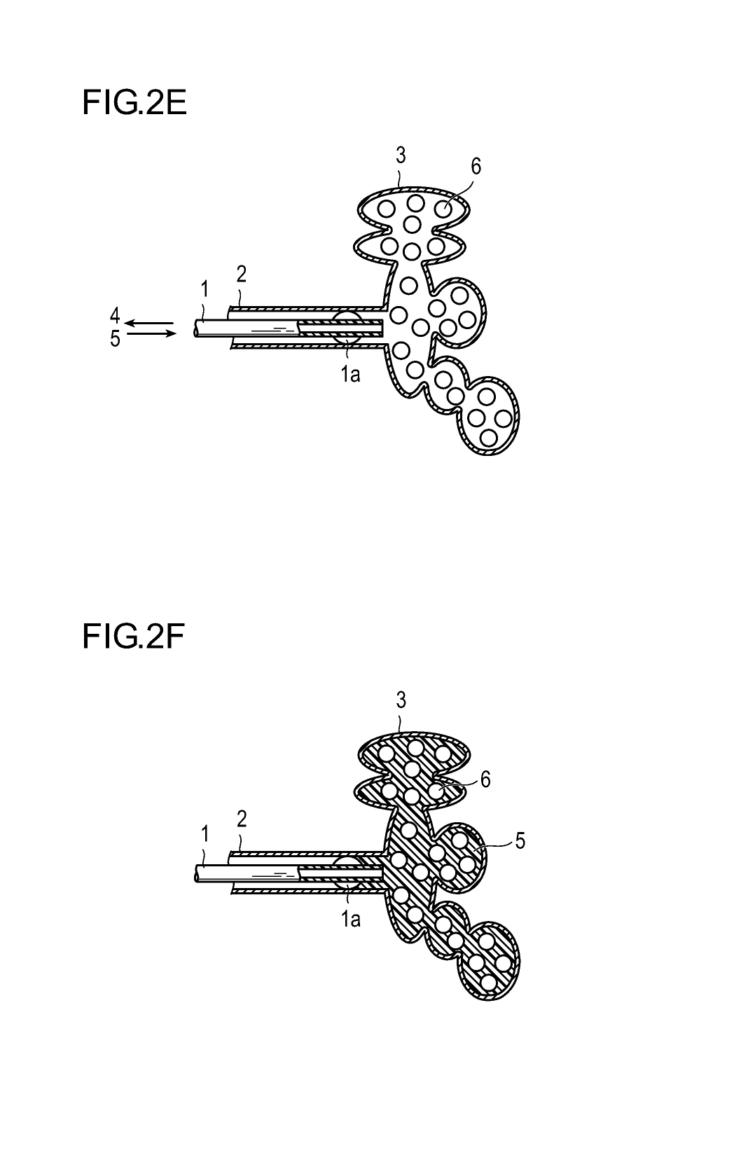

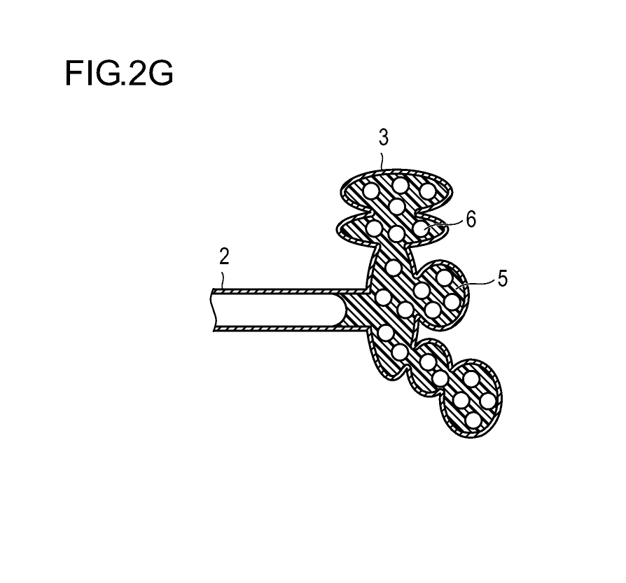

FIGS. 1A to 1F and FIGS. 2A to 2G are schematic sectional views showing the procedures of steps in preferred methods according to embodiments of the present invention. The steps will be described in detail below, referring to FIGS. 1A to 1F and FIGS. 2A to 2G, but the steps are not to be restricted to the following modes.

### 1. Step (a)

In this step, as shown in FIG. 1A and FIG. 2A, a catheter 1 is inserted into a bronchus or bronchiole 2 leading to a respiratory region including pulmonary alveoli or alveolar sac suffering from emphysema (hereinafter referred also to simply as “alveolar parenchyma suffering from emphysema”) 3.

Here, the catheter is not particularly limited but may be of any structure insofar as it has a balloon. The catheter is appropriately selected according to the diameter (the number of branching) of the bronchus or bronchiole into which it is to be inserted. To be more specific, it may be selected from any known medical catheters for the respiratory system, circulatory system, and digestive system. Besides, the catheter is not limited in the number of its lumens. The number of lumens is properly selected according to the number of materials to be used in the step (d), which will be detailed below, the presence or absence of the discharge of stagnant gas, etc.

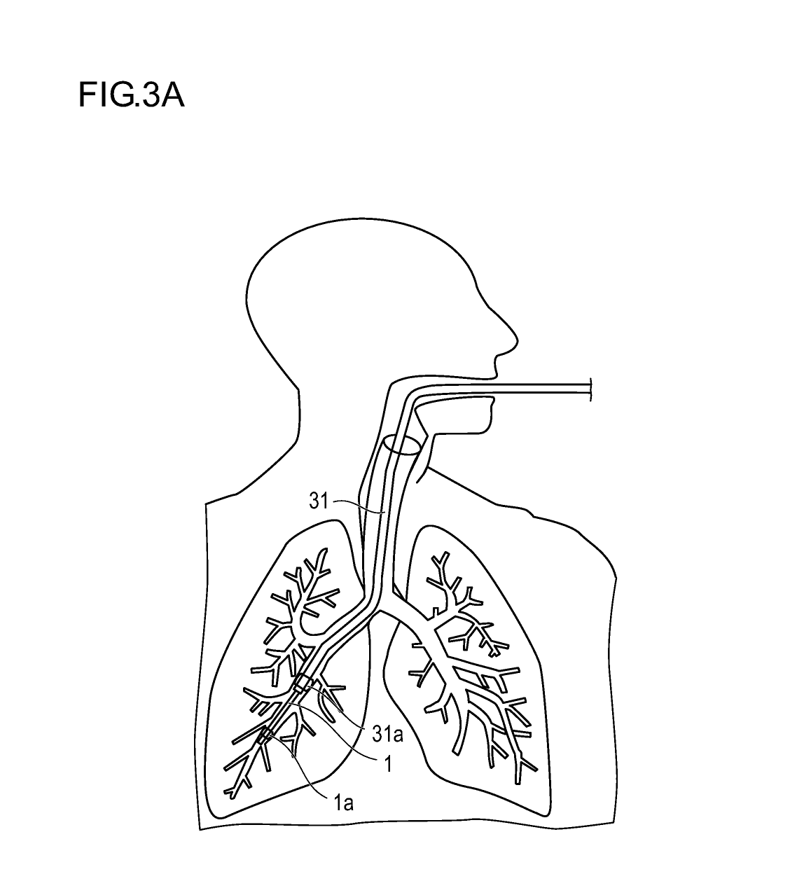

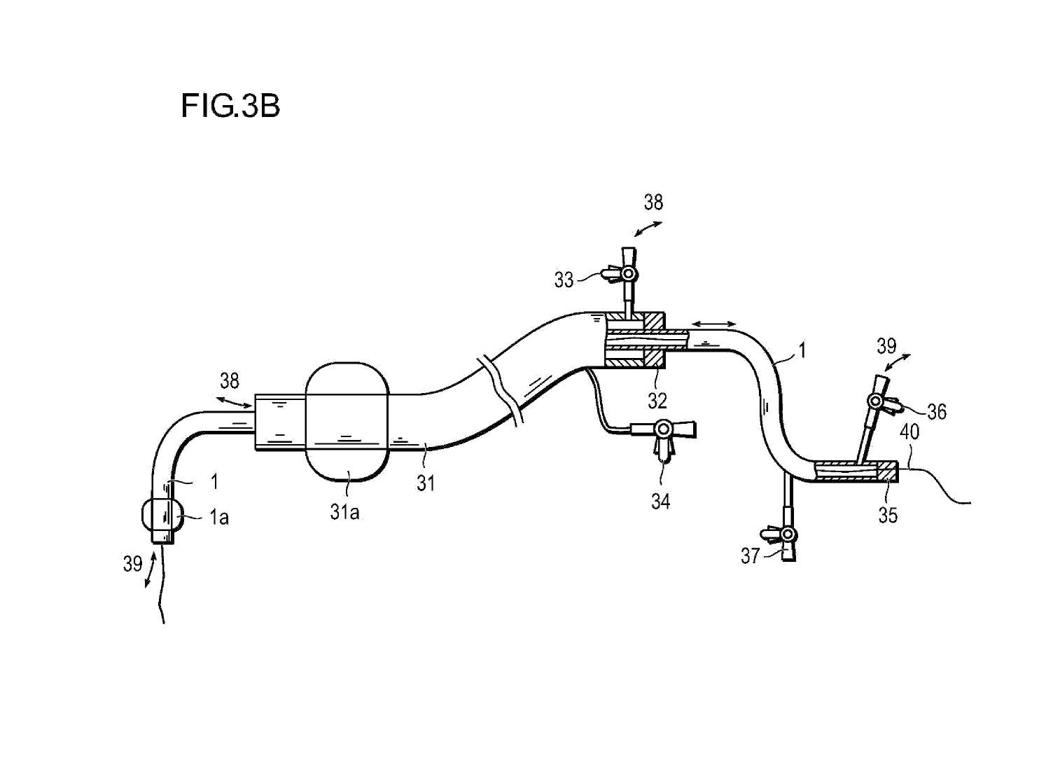

In inserting the catheter 1 into the alveolar parenchyma 3 suffering from emphysema, it may be inserted via a sheath 31 disposed on the more proximal side, as shown in FIG. 3A. The sheath 31 is not specifically restricted in structure, and it may or may not have a balloon. It is preferable, however, that the sheath 31 has a balloon 31a capable of closing the bronchus or bronchiole. In this case, the positions at which the balloon 31a attached to the sheath 31 and the balloon 1a attached to the catheter 1 are disposed in the bronchus or bronchiole are not particularly restricted. Preferably, the balloon 31a attached to the sheath 31 is disposed in the bronchus; while the balloon 1a attached to the catheter 1 is disposed on the more peripheral side in the bronchus, particularly in the bronchiole. The closure of the bronchus or bronchiole with the balloon 31a promises an enhanced air-tightness on the distal side of the sheath, which permits the alveolar parenchyma suffering from emphysema to be treated more effectively by use of the catheter. In addition, both the balloons 1a and 31a may be used to close different parts of the bronchus or bronchiole. This makes it possible to easily and individually control the pressure between the balloons 1a and 31a (for example, in the normal alveolar parenchyma) and the pressure on the peripheral side of the balloon 1a (for example, in the alveolar parenchyma suffering from emphysema).

By closing the bronchus or bronchiole with the balloon 31a, it is possible on the proximal side of the balloon 31a to maintain ventilation through addition of respiratory pressures, thereby carrying out an efficient and safe treatment. Here, the method for dilation and contraction of the balloon 31a is not specifically restricted, and may be by use of a three-way stopcock 34 provided on the proximal side of the sheath 31.

Further, the pressure on the distal side of the balloon 31a attached to the sheath 31 is kept constant, whereby operations on the distal side relative to the catheter 1 can be carried out stably. In an example, the bronchus or bronchiole is closed with the balloon 31a and the pressure on the distal side relative to the sheath 31 is reduced, whereby the adhesion of the wall of the bronchus or bronchiole to the balloon 1a attached to the catheter 1 is enhanced. In addition, the flow of gas from the bypass to the distal side relative to the catheter 1 is prevented by the balloon 1a, whereby decompression on the distal side relative to the catheter is facilitated. Besides, in injecting a drug such as a hardening agent at a constant pressure into the space on the distal side of the catheter 1, the pressure on the distal side relative to the sheath 31 is kept constant and lower than the drug injection pressure, whereby efficient drug delivery is secured. Here, the methods for controlling the pressure on the distal (peripheral) side relative to the sheath 31 and the pressure on the distal (peripheral) side relative to the catheter 1 are not particularly restricted. One specific example of the method for pressure control is shown in FIG. 3B. A sealing stopcock 32 is provided on the proximal side of the sheath 31, and the catheter 1 is inserted into the sheath 31 via the sealing stopcock 32. With the sealing stopcock 32 provided in this manner, the alveolar parenchyma on the distal (peripheral) side relative to the sheath 31 can be made a closed system, so that pressure control in that part can be performed easily. In addition, where a three-way stopcock 33 is provided at a proximal portion of the sheath 31 and a gas 38 is introduced or sucked in through the three-way stopcock 33, it is possible to control the pressure inside the alveolar parenchyma on the distal (peripheral) side of the sheath 33. Control of the pressure on the distal (peripheral) side relative to the catheter 1 can also be conducted in the same manner. To be more specific, as shown in FIG. 3B, a sealing stopcock 35 is provided on the proximal side of the catheter 1. Arrangement of the sealing stopcock 35 makes it possible to render the space inside the alveolar parenchyma on the distal (peripheral) side of the catheter 1a closed system, thereby facilitating pressure control in that part. In addition, where a three-way stopcock 36 is provided on the proximal side of the catheter 1 and a gas or liquid 39 is introduced or sucked in through the three-way stopcock 36, it is possible to control the pressure inside the alveolar parenchyma on the distal (peripheral) side relative to the catheter 1. Besides, the methods of dilating and contracting the balloon la are not particularly limited, and may be accomplished by use of a three-way stopcock 37 provided on the proximal side of the catheter 1. Further, in order to facilitate the insertion of the catheter 1 to a desired position more easily, the catheter 1 may have a lumen for introducing a guide wire 40.

For example, that catheter 1 having the balloon 1a for closing the bronchus is used which is a catheter having a lumen provided with openings on the distal side and the proximal side and capable of feeding a liquid toward the distal side, or which is a PTCA catheter of the OTW type used in treatment of stenosis of vascular lumen in a cardiovascular region. Here, the catheter may be a commercial one. For example, there may be a microcatheter (e.g., FINECROSS (registered trademark), made by TERUMO CORPORATION), a PTCA catheter (e.g., Ryujin Plus OTW (registered trademark), made by TERUMO CORPORATION), etc. which are used for passing a guide wire into stenosis of a vascular lumen in a cardiovascular region. Here, the catheter may be inserted through a working lumen of a bronchoscope into the bronchial lumen, use of the bronchoscope is not indispensable, insofar as the catheter can be disposed in an arbitrary location. In addition, the outside diameter of the catheter 1 or the balloon 1a upon expansion is not specifically limited but is appropriately selected according to the diameter of the bronchus or bronchiole. Specifically, the outside diameter of the balloon 1a upon dilation is desirably slightly greater than the inside diameter of the bronchus or bronchiole 2 communicating with the tissue of arbitrary alveolar sacs (air sacs) or pulmonary alveoli in which the catheter 1 is to be inserted and which is to be coated. More desirably, the outside diameter [Y (mm)] of the balloon 1a upon dilation is about 1 to 2 times the inside diameter [X (mm)] of the bronchus or bronchiole 2. In this case, the bronchus or bronchiole formed from highly elastic smooth muscles can be pressed against the catheter or balloon part, without being damaged excessively. In addition to the foregoing, it is possible to enhance the removal efficiency in the case of discharging a film-forming agent 4, which will be described later from the respiratory region.

In this step, a guide wire may be inserted in the lumen (e.g., a liquid feeding lumen) of the catheter, thereby guiding the catheter into the bronchus or bronchiole 2. This ensures that operation can be carried out while maintaining a positional relationship in which the distal end of the guide wire is located on the peripheral side relative to the distal end of the catheter. Therefore, a catheter distal portion can be guided into the vicinity of a tissue of the alveolar sacs (air sacs) or pulmonary alveoli on the peripheral side relative to the bronchus or bronchiole 2. Here, the guide wire to be used may be any of known medical guide wires for the respiratory system, circulatory system, or digestive system. The outside diameter, etc. of the guide wire can be properly selected according to the size of the lumen in the catheter used therewith, etc. Specific examples of the guide wire (hereinafter referred to as GW) which can be used include those used for treatment of heart blood vessels, such as Runthrough (registered trademark) (made by TERUMO CORPORATION; outside diameter: 0.014 inch).

A member having contrast is preferably disposed at a distal portion of the guide wire or a distal end of the catheter. Observation under X-ray fluoroscopy ensures that the positions of the distal ends of the guide wire and the catheter protruding from the distal end of the endoscope can be grasped. This permits the guide wire and the catheter to be guided to a respiratory region including pulmonary alveoli or alveolar sacs suffering from emphysema which has preliminarily located by X-ray fluoroscopy or CT scanning. In this case, the guide wire is pulled out after the X-ray fluoroscopy reveals that the distal end of the catheter has reached the desired position. The operation is preferably carried out while maintaining the positional relationship in which the distal end of the guide wire is disposed on the peripheral side relative to the distal end of the catheter. In addition, the distal end of the catheter preferably has a reticulate or perforated structure so that its adhesion to the inner wall of the respiratory region including the pulmonary alveoli and alveolar sacs can be restrained or prevented.

### 2. Step (b)

In this step, as shown in FIGS. 1B and 2B, the balloon 1a is dilated to close the bronchus or bronchiole 2. As a result of this operation, the inside of the alveolar parenchyma suffering from emphysema which is accompanied by no bypass is made to be a closed system, except for openings communicating with the bronchi or bronchioles. Therefore, when the alveolar parenchyma is shrunk in the subsequent step (c), there would be little or no leakage of air from the alveolar parenchyma suffering from emphysema. This ensures that air in the closed system can be removed assuredly, and the volume of the alveolar parenchyma can be reduced efficiently.

Here, the position where the balloon is disposed is not specifically restricted. For instance, the balloon may be disposed at the distal (peripheral) end of the catheter, or may be disposed on the trachea (proximal) side of the distal (peripheral) end of the catheter. In the case where the catheter distal end is located in the bronchus, the balloon is preferably disposed at the catheter in such a manner as not to come beyond the branching on the proximal side of the bronchus. This permits the closed system of the alveolar parenchyma suffering from emphysema to be achieved more securely.

The balloon 1a may contain a contrast agent which is opaque to X-rays or the like, or such a contrast agent may be injected into the balloon at the time of dilating the balloon. This permits the degree of dilation of the balloon to be confirmed under X-ray fluoroscopy, so that the bronchus or bronchiole 2 can be closed with the balloon 1a more reliably and easily. Here, the contrast agent is not specifically restricted insofar as it is opaque to radiations, and any of known contrast materials can be used. Specific examples of the contrast material which can be used include: iodine, barium, bismuth, boron, bromine, calcium, gold, platinum, silver, iron, manganese, nickel, gadolinium, dysprosium, tungsten, tantalum, stainless steel, Nitinol, their compounds such as barium sulfate, and their solutions/dispersions (e.g., physiological saline); amidotrizoic acid (3,5-diacetamino-2,4,6-triiodobenzoic acid), sodium meglumine amidotrizoate, meglumine amidotrizoate, sodium iotalamate, meglumine iotalamate, meglumine iotroxate, iotrolan, ioxaglic acid, ioxilan, iopamidol, iopromide, iohexyl, ioversol, iomeprol; and iodine addition products of ethylesters of the fatty acids obtained from poppyseed oil (e.g., Lipiodol™, a poppy seed oil having carbon atom iodized). These radiopaque materials may be used either singly or in combination of two or more of them. Or, the balloon may be provided with a contrast agent layer formed by use of the above-mentioned contrast agent as a base material. Incidentally, the degree of dilation (expansion) of the balloon is not specifically restricted, insofar as it ensures that the bronchus or bronchiole 2 can be closed assuredly and that the bronchus or bronchiole 2 is not damaged.

### 3. Step (c)

In this step, the pulmonary alveoli or alveolar sacs on the downstream side of the bronchus or bronchiole closed in the above-mentioned step (b) are shrunk. By this step, the alveolar parenchyma suffering from emphysema is swiftly shrunk, whereby stagnant air in the alveolar parenchyma suffering from emphysema can be removed efficiently. Incidentally, in this step, the normal alveolar parenchyma may also be shrunk simultaneously. Particularly in the case where the catheter has been inserted to reach the bronchiole, however, the proportion of the normal alveolar parenchyma based on the whole alveolar parenchyma is very low, so that the influence of the shrinkage and the influence of the hardening by the hardening agent in the subsequent step are negligible.

While the alveolar parenchyma suffering from emphysema is shrunk in this step, the method for shrinking the alveolar parenchyma (pulmonary alveoli or alveolar sacs) suffering from emphysema is not specifically restricted. For instance, the methods as set forth in the following (c-1) and (c-2) are preferably used:

(c-1) a method in which a gas remaining in the respiratory region including the pulmonary alveoli or alveolar sacs is removed by suction through the catheter; and

(c-2) a method in which a gas-absorbing agent for absorbing air present in the pulmonary alveoli or alveolar sacs is injected into the pulmonary alveoli or alveolar sacs.

While the methods as set forth in (c-1) and (c-2) above will be described in detail below, the present invention is not to be restricted to or by these methods.

3-1. Step (c-1)

In this method, as shown in FIG. 1C, a residual gas (air) 4 in the respiratory region including the pulmonary alveoli or alveolar sacs is removed by suction via the catheter 1. In this instance, the inside of the alveolar parenchyma 3 suffering from emphysema has been made a closed system except for openings communicating with the bronchi or bronchioles, in the above-mentioned step (b). Therefore, it is ensured that when the gas 4 remaining in the alveolar parenchyma is sucked in this step, there is little or no leakage of air from the alveolar parenchyma 3 suffering from emphysema. Therefore, it is possible to securely remove the residual gas (air) in the alveolar parenchyma 3 suffering from emphysema and to shrink the alveolar parenchyma suffering from emphysema efficiently and speedily; in other words, it is possible to reduce the volume of the alveolar parenchyma efficiently and speedily. Here, it suffices for the removal by suction of the residual gas 4 in the alveolar parenchyma 3 suffering from emphysema to be finished at the time when the suction of the residual gas becomes impossible. Besides, it is preferable that the balloon 1a should be in the dilated state during this step. This ensures that inflow of air from the bronchus or bronchiole on the more upstream side can be prevented. Consequently, the residual gas 4 in the alveolar parenchyma 3 suffering from emphysema can be securely removed by suction through the catheter 1 so as to reduce the internal volume of the alveolar parenchyma 3. In addition, this operation results in that the bronchus or bronchiole 2 on the peripheral side relative to the balloon part collapses (decreases in diameter of the bronchiole 2), and the inside wall of the bronchus or bronchiole 2 is pressed against (brought into firm contact with) the surface of the balloon part. Therefore, a negative-pressure state is liable to be kept inside the alveolar parenchyma 3 suffering from emphysema. Incidentally, the term “respiratory region” as used in this specification is a generic term for respiratory organs on the distal side relative to the bronchi including respiratory bronchiole and two alveoli. To be more specific, the respiratory region include bronchi, bronchioles, terminal bronchioles, respiratory bronchioles, alveolar ducts, pulmonary alveoli, alveolar sacs, pulmonary veins, and pulmonary arteries, and preferably includes respiratory bronchioles, alveolar ducts, pulmonary alveoli, alveolar sacs, and pulmonary veins.

The rate of suction for the residual gas 4 present in the alveolar parenchyma 3 suffering from emphysema is not specifically restricted insofar as it is such a rate as to remove the residual gas (air) in the alveolar parenchyma suffering from emphysema and not to damage the alveolar parenchyma suffering from emphysema. Besides, it suffices for the removal by suction of the residual gas 4 in the alveolar parenchyma 3 suffering from emphysema to be finished at the time when suction of the residual gas 4 becomes impossible.

3-2. Step (c-2)

In this method, as shown in FIG. 2C, a gas-absorbing agent 6 for absorbing air present in the pulmonary alveoli or alveolar sacs 3 is injected through the catheter 1 into the respiratory region 2, thereby filling the pulmonary alveoli or alveolar sacs (alveolar parenchyma suffering from emphysema) 3 with the gas-absorbing agent 6. Incidentally, at the time of filling with the gas-absorbing agent 6, it is preferable to dilate the balloon 1a so as to achieve sealing between the catheter 1 and the inside walls of the bronchus or bronchiole 2 (FIG. 2C). This permits the gas-absorbing agent 6 to be loaded into (to fill) the inside of the pulmonary alveoli or alveolar sacs 3 more assuredly, without flowing backward. The gas-absorbing agent 6 thus loaded absorbs air present in the alveolar parenchyma 3 suffering from emphysema, thereby shrinking the alveolar parenchyma suffering from emphysema, whereby the lung volume of the alveolar parenchyma suffering from emphysema is reduced (FIG. 2D). Here, as above-mentioned, the alveolar parenchyma 3 suffering from emphysema has been made to be a closed system except for the openings communicating with the bronchus or bronchiole in the above-mentioned step (b). In the present step, therefore, the volume of the alveolar parenchyma can be reduced efficiently and speedily. Incidentally, the gas-absorbing agent 6 may be removed by suction or the like. However, the alveolar parenchyma thus shrunk would not function as pulmonary alveoli or alveolar sacs anymore, and, accordingly, as shown in FIG. 2D, the gas-absorbing agent 6 may not necessarily be removed.

The gas-absorbing agent 6 is not particularly limited, insofar as it absorbs air present in the alveolar parenchyma suffering from emphysema. Examples of the gas-absorbing agent 6 include those gas-absorbing agents which contain, as main ingredient: silica, ceramics, porous ceramics, magnesia, titania, calcium silicate, activated carbon; iron powders such as pure iron powder, cast iron powder, steel powder, reduced iron powder, sprayed iron powder, spongy iron powder, electrolytic iron powder, iron alloy powders, etc., aluminum powder, magnesium powder, silicon fine powder; L-ascorbic acid and isoascorbic acid (erythorbic acid) and their alkali metal salts and alkaline earth metal salts; polyhydric alcohols such as glycerin, ethylene glycol, propylene glycol, etc.; phenol compounds such as catechol, resorcin, hydroquinone, gallic acid, pyrogallol, tocopherol, etc.; and reducing sugars such as glucose, fructose, sorbitol, xylose, etc. These gas-absorbing agents may be used either singly or in combination of two or more of them. Among the above-mentioned gas-absorbing agents, preferred are iron powders, ceramics, and porous ceramics, and more preferred are ceramics and porous ceramics. These are excellent in safety.

Where the iron powders among the above-mentioned gas-absorbing agents are used, an oxidation-accelerating substance is preferably used together. The use of the oxidation-accelerating substance promises an enhanced oxygen-absorbing function. Here, the oxidation-accelerating substance is not specifically restricted, and examples thereof include alkali metal or alkaline earth metal halides such as NaCl, CaCl₂, MgCl₂, etc., halides of ion exchange resins, hydrochloric acid and hypochlorite. Besides, the amount of the oxidation-accelerating substance(s) is preferably 0.01 to 20 parts by weight based on 100 parts by weight of the iron powder.

The amount of the gas-absorbing agent introduced into the alveolar parenchyma suffering from emphysema is not specifically restricted, insofar as it makes it possible to sufficiently absorb air present in the pulmonary alveoli or alveolar sacs and to reduce the volume of the alveolar parenchyma suffering from emphysema. The amount can be appropriately selected in consideration of the volume of the alveolar parenchyma suffering from emphysema. Or, the injection of the gas-absorbing agent may be stopped upon detection of a rise in the injection pressure for the gas-absorbing agent.

The retention time for the thus introduced gas-absorbing agent in the alveolar parenchyma suffering from emphysema is not particularly limited, and is preferably in the range of one to 10 minutes. Such a length of time ensures that the gas-absorbing agent sufficiently absorbs air present in the pulmonary alveoli or alveolar sacs, whereby the volume of the alveolar parenchyma suffering from emphysema can be reduced.

Of the above-mentioned (c-1) and (c-2), preferred is (c-1), taking into account ease of operation and the like.

### 4. Step (d)

In this step, as shown in FIGS. 1D and 2E, a hardening agent 5 is injected through the catheter 1 into the respiratory region, particularly into the alveolar parenchyma 3 suffering from emphysema that has been shrunk in the above-mentioned step (c), to fill up the alveolar parenchyma suffering from emphysema with the hardening agent 5, followed by hardening the hardening agent 5 (FIGS. 1E and 2F). This permits the alveolar parenchyma suffering from emphysema to be maintained in the shrunk state, without re-expansion. Therefore, the volume of the alveolar parenchyma suffering from emphysema can be reduced efficiently. In addition, it is possible to maintain the shrunk state of the alveolar parenchyma suffering from emphysema, to reduce efficiently the volume of the alveolar parenchyma, and to maintain the thus reduced volume by respiration. Therefore, it is possible to alleviate or suppress the overexpansion of the lung, which is one of the causes of weakening of the patient by emphysema or occlusion of the bronchi. Besides, it is possible to restore the size of the alveolar parenchyma suffering from emphysema to its original size or less, thereby suppressing or preventing the compression or occlusion of the surrounding bronchi by the surrounding alveolar parenchyma. In addition to the foregoing, the method pertaining to the present invention resides in therapy through the use of a catheter and does not need a surgical treatment; therefore, the present method promises an alleviated burden on the patient. In this instance, the balloon 1a is preferably in its dilated state (FIGS. 1D and 2E). This permits the hardening agent to be efficiently injected into the alveolar parenchyma 3 suffering from emphysema, without flowing back toward the bronchus side.

In the present step, as shown in FIGS1D and 2E, injection of the hardening agent 5 through the catheter 1 into the respiratory region is preferably carried out while maintaining a reduced pressure inside the respiratory region, particularly inside the pulmonary alveoli or alveolar sacs (alveolar parenchyma suffering from emphysema) 3, by sucking the residual gas 4 in the alveolar parenchyma 3 suffering from emphysema. By this operation, the hardening agent 5 can be speedily and efficiently injected into the alveolar parenchyma 3 suffering from emphysema. In addition, even in the case where foams are present in the alveolar parenchyma 3 suffering from emphysema, the foams are removed by suction through the catheter. Accordingly, the inside of the alveolar parenchyma 3 can be filled up with the hardening agent more assuredly, and gas exchange in the alveolar parenchyma 3 can be suppressed or prevented more securely. In addition to the foregoing, since the inside of the alveolar parenchyma 3 is decompressed, expansion of the alveolar parenchyma 3 is restrained or prevented, so that the shrunk state of the alveolar parenchyma 3 can be maintained. Or, depending on the degree of decompression, the alveolar parenchyma 3 can also be further shrunk. Here, the rate of suction of the residual gas 4 in the alveolar parenchyma 3 suffering from emphysema (the degree of decompression) is not particularly restricted, and it is preferable that the suction rate is substantially the same as the injection rate (injection pressure) of the hardening agent. This permits the hardening agent to be smoothly injected into the alveolar parenchyma 3. Besides, even in the case where foams are remained in the alveolar parenchyma 3, the foams can be removed.

Here, the hardening agent is not specifically restricted, insofar as it is a safe material which is hardened at a temperature of not more than 45° C., particularly in the vicinity of the body temperature. Preferably, the hardening agent has at least one of the following characteristic properties: (a) to be safe to the patient; (b) to cause little or no damage to the tissues; (c) to be able to be hardened at a temperature close to the patient's body temperature (about 35 to 42° C.); (d) to be able to maintain a shape upon hardening, without undergoing shrinkage or expansion; (e) to be able to be hardened in 60 minutes, preferably in 30 minutes, more preferably in 20 minutes, after injection; and (f) to permit use of water or oils and fats such as olive oil, castor oil, etc. as a solvent. Specific examples of the hardening agent include: (i) gelatin, agar, starch; (ii) combinations of an aromatic diepoxide resin or aliphatic diepoxide resin with an amine compound; (iii) combinations of water with a material capable of hardening by reaction with water; (iv) combinations of divalent metal ions with a material capable of hardening by reaction with the divalent metal ions; and (v) combinations of a polymeric electrolyte having a negative charge with a polymeric electrolyte having a positive charge.

In (ii) above, the aromatic diepoxide resin is not particularly restricted, and examples thereof include diglycidyl ether of bisphenol A, and diglycidyl ether of bisphenol F. The aliphatic diepoxide resin is not specifically restricted, and examples thereof include diepoxide resins which are alkane diols of glycidyl ethers, such as pentane diol of glycidyl ether, butane diol of glycidyl ether, and propane diol of glycidyl ether. In addition, the amine compound is not specifically restricted, and examples thereof include 1,3-diaminopropane, diethylenetriamine, triethylenetetramine, N-aminoethylpiperadinenonyl/phenol, and dialkylamine compounds represented by the formula: H₂N—(R—NH)ₓ—R—NH₂ (where R groups are independently straight chain or branched chain alkyl groups of 2 to 10 carbon atoms, preferably 2 to 5 carbon atoms, and x is 0, 1, or 2). Among these hardening agents of (ii) above, preferred are those including about 60 to 80 wt %, more preferably about 65 to 75 wt % of the aromatic diepoxide resin and/or aliphatic diepoxide resin and about 20 to 40 wt %, more preferably about 25 to 35 wt % of the amine compound [the total amount of the aromatic diepoxide resin and/or aliphatic diepoxide resin and the amine compound is 100 wt %]. Besides, particularly preferred are those including about 45 to 52 wt % of the aromatic diepoxide resin, about 19 to 23 wt % of the aliphatic diepoxide resin, about 20 to 29 wt % of the dialkylamine compound, and about 4 to 9 wt % of the N-aminoethylpiperadinenonyl/phenol [the total amount of the aromatic diepoxide resin, the aliphatic diepoxide resin, the dialkylamine compound and the N-aminoethylpiperadinenonyl/phenol is 100 wt %].

In (iii) above, the material capable of hardening by reaction with water is not particularly restricted, insofar as it starts reaction (hardening) in the presence of water. Specifically, a cyanoacrylate monomer is preferably used. In this case, the cyanoacrylate monomer polymerizes upon contact with water or moisture, to be polycyanoacrylate. Specific examples of the cyanoacrylate monomer include: alkyl and cycloalkyl α-cyanoacrylates such as methyl α-cyanoacrylate, ethyl α-cyanoacrylate, propyl α-cyanoacrylate, butyl α-cyanoacrylate, pentyl α-cyanoacrylate, hexyl α-cyanoacrylate, heptyl α-cyanoacrylate, octyl α-cyanoacrylate, cyclohexyl α-cyanoacrylate, etc; alkenyl and cycloalkenyl α-cyanoacrylates such as allyl α-cyanoacrylate, methallyl α-cyanoacrylate, cyclohexenyl α-cyanoacrylate, etc.; alkynyl α-cyanoacrylates such as propargyl α-cyanoacrylate, etc.; aryl α-cyanoacrylates such as phenyl α-cyanoacrylate, toluoyl α-cyanoacrylate, etc.; hetero-atom-containing methoxyethyl α-cyanoacrylate, ethoxyethyl α-cyanoacrylate, and furfuryl α-cyanoacrylate; and silicon-containing trimethylsilylmethyl α-cyanoacrylate, trimethylsilylethyl α-cyanoacrylate, trimethylsilylpropyl α-cyanoacrylate, dimethylvinylsilylmethyl α-cyanoacrylate, etc. These α-cyanoacrylates may be used either singly or in combination of two or more of them. Among these, preferred are cyclohexyl α-cyanoacrylate, heptyl α-cyanoacrylate, octyl α-cyanoacrylate, cyclohexenyl α-cyanoacrylate, and the like. A polymerized hardened product of a cyanoacrylate with such a long ester side chain is flexible, and gives little damage to the alveolar parenchyma tissue. Incidentally, in the case of using the material capable of hardening by reaction with water, a plasticizer may further be used in addition to that material. The use of the plasticizer imparts flexibility to the polymerized hardened product, whereby damage to the alveolar parenchyma tissue can be further suppressed or prevented.

In (iv) above, the combination of divalent metal ions and a material capable of hardening by reaction with the divalent metal ions is not specifically restricted, insofar as the mixing of the two components results in hardening. Specific examples of the combination include combinations of alginic acid with solutions prepared by dissolving in water a compound which produces divalent metal ions such as calcium ions, magnesium ions, barium ions, etc. in a solution, such as calcium chloride, calcium hydrogenphosphate, calcium dihydrogenphosphate, tricalcium phosphate, calcium sulfate, calcium hydroxide, magnesium chloride, barium chloride, etc. Among these, preferred is a combination of alginic acid with a compound producing calcium ions in a solution, particularly, a combination of an aqueous solution of sodium alginate and an aqueous solution of calcium chloride hydrate. In this case, alginic acid and the calcium compound undergo gelation by a crosslinking reaction, thereby showing efficient hardening in the alveolar parenchyma suffering from emphysema.

In (v) above, the polymeric electrolyte having a negative charge and the polymeric electrolyte having a positive charge react with each other to form an ion complex, resulting in hardening.

Here, the polymeric electrolyte having a negative charge is not particularly restricted, insofar as it has at least one, preferably two or more anionic groups. Examples of the polymeric electrolyte having a negative charge include: proteins such as collagen, albumin, fibronectin, gelatin which is a modified product of collagen, etc.; polyamino acids; synthetic polypeptides which are synthesized artificially; polysaccharides such as heparin, chitin, hyaluronic acid, chondroitin, pectin, agarose, glycosaminoglycan, cellulose, starch, etc.; artificially synthesized polysaccharides; and synthetic polymers such as polyethylene glycol, polypropylene glycol, polyethylene/polypropylene copolymer, etc. Here, the weight average molecular weight of the synthetic polymer is not particularly limited, and may be about 10,000 to about 1,000,000, preferably about 100,000 to about 700,000, more preferably about 200,000 to about 500,000. The above-mentioned polymeric electrolytes may be used either singly or in combination of two or more of them. Among these polymeric electrolytes, preferred are gelatin, heparin, hyaluronic acid, chondroitin, pectin, agarose, glycosaminoglycan, and more preferred are gelatin, heparin, and hyaluronic acid.

Alternatively, the polymeric electrolyte having a negative charge may be obtained by polymerization of a monomer having a negative charge. Here, nonlimitative examples of the monomer having a negative charge include monomers which each have at least one functional group selected from among a sulfo group (—SO₃H), a carboxyl group (—COOH), a phosphonic acid group (—PO₃H₂), etc.

Among the above-mentioned, the monomer having the sulfo group (—SO₃H) is not particularly restricted, and examples thereof include vinylsulfonic acid (ethylenesulfonic acid), 2-propensulfonic acid, 3-butenesulfonic acid, 4-pentenesulfonic acid, sulfomethyl(meth)acrylate, 2-sulfoethyl(meth)acrylate, 3-sulfopropyl(meth)acrylate, 2-methyl-3-sulfopropyl(meth)acrylate, 4-sulfobutyl(meth)acrylate, N-(2-sulfoethyl) 4-sulfobutyl(meth)acrylate, 2-(meth)acrylamido-2-methylpropanesulfonic acid, N-(2-sulfoethyl)(meth)acrylamide, N-(1-methyl-2-sulfoethyl)(meth)acrylamide, N-(2-methyl-3-sulfopropyl)(meth)acrylamide, N-(4-sulfobutyl)(meth)acrylamide, 10-sulfodecyl(meth)acrylate, styrenesulfonic acid, (meth)allyl sulfonate, allylsulfonic acid, 3-(meth)acryloxy-2-hydroxypropyl sulfonate, 3-(meth)acryloxy-2-hydroxypropyl sulfophenyl ether, 3-(meth)acryloxy-2-hydroxypropyloxysulfobenzoate, 4-(meth)acryloxybutyl sulfonate, (meth)acrylamidomethylsulfonic acid, (meth)acrylamidoethylsulofonic acid, (meth)acrylamide 2-methylpropanesulfonate.

In addition, the monomer having the carboxyl group is not particularly restricted, and examples thereof include (meth)acrylic acid, maleic acid, fumaric acid, glutaconic acid, itaconic acid, crotonic acid, sorbic acid, cinnamic acid, N-(meth)acryloylglycine, N-(meth)acryloylaspartic acid, N-(meth)acryloyl-5-aminosalicylic acid, 2-(meth)acryloyloxyethyl hydrogensuccinate, 2-(meth)acryloyloxyethyl hydrogenphthalate, 2-(meth)acryloyloxyethyl hydrogenmaleate, 6-(meth)acryloyloxyethylnaphthalene-1,2,6-tricarboxylic acid, O-(meth)acryloyltyrosine, N-(meth)acryloyltyrosine, N-(meth)acryloylphenylalanine, N-(meth)acryloyl-p-aminobenzoic acid, N-(meth)acryloyl-o-aminobenzoic acid, p-vinylbenzoic acid, 2-(meth)acryloyloxybenzoic acid, 3-(meth)acryloyloxybenzoic acid, 4-(meth)acryloyloxybenzoic acid, N-(meth)acryloyl-5-aminosalicylic acid, N-(meth)acryloyl-4-aminosalicylic acid, etc.

The monomer having the phosphonic acid group is not specifically restricted, and examples thereof include phosphooxyethyl(meth)acrylate, 3-(meth)acryloxypropyl-3-phosphonopropionate, 3-(meth)acryloxypropylphosphonoacetate, 4-(meth)acryloxybutyl-3-phosphonopropionate, 4-(meth)acryloxybutylphosphonoacetate, 5-(meth)acryloxypentyl-3-phosphonopropionate, 5-(meth)acryloxypentylphosphonoacetate, 6-(meth)acryloxyhexyl-3-phosophonopropionate, 6-(meth)acryloxyhexylphosphonoacetate, 10-(meth)acryloxydecyl-3-phosphonopropionate, 10-(meth)acryloxydecylphosphonoacetate, 2-(meth)acryloxyethyl phenylphosphonate, 2-(meth)acryloyloxyethylphosphonic acid, 10-(meth)acryloyloxydecylphosphonic acid, N-(meth)acryloyl-ω-aminopropylphosphonic aid, etc.

The above-mentioned monomers may be used either singly or in combination of two or more of them.

Besides, the polymeric electrolyte having a positive charge is not specifically restricted, insofar as it has at least one, preferably two or more cationic groups. Examples include: organic compounds having an N,N-dimethylaminoalkyl group in a side chain thereof; polyethyleneimine, etc. Here, the weight average molecular weight of the above-mentioned polymeric electrolyte is not particularly limited, and is preferably 10,000 to 1,000,000, more preferably 100,000 to 500,000. These polymeric electrolytes may be used either singly or in combination of two or more of them. Among these polymeric electrolytes, preferred are poly(N,N-dimethylaminopropylacrylamide) having a weight average molecular weight of about 10,000 to about 1,000,000, poly(N,N-dimethylaminoethylacrylamide) having a weight average molecular weight of about 10,000 to about 1,000,000, and polyethyleneimine having a weight average molecular weight of about 10,000 to about 1,000,000. More preferred among these are poly(N,N-dimethylaminopropylacrylamide) having a weight average molecular weight of about 10,000 to about 500,000, poly(N,N-dimethylaminoethylacrylamide) having a weight average molecular weight of about 10,000 to about 500,000, and polyethyleneimine having a weight average molecular weight of about 10,000 to about 500,000 (particularly, about 100,000).

Alternatively, the polymeric electrolyte having the positive charge may be obtained by polymerization of a monomer having a positive charge. Here, nonlimitative examples of the monomer having a positive charge include monomers having at least one functional group selected from among the amino group (—NH₂), the imino group (═NH, —NH—), the imidazoyl group, the pyridyl group, and the like.

Among the above-mentioned, the monomer having the amino group is not specifically restricted, and examples thereof include (meth)allylamine, aminoethyl(meth)acrylate, aminopropyl(meth)acrylate, dimethylaminoethyl(meth)acrylate, diethylaminoethyl(meth)acrylate, methylethylaminoethyl(meth)acrylate, dimethylaminopropyl(meth)acrylate, dimethylaminostyrene, diethylaminostyrene, morpholinoethyl(meth)acrylate, lysine, etc.

The monomer having the imino group is not specifically restricted, and examples thereof include N-methylaminoethyl(meth)acrylate, N-ethylaminoethyl(meth)acrylate, N-t-butylaminoethyl(meth)acrylate, ethyleneimine, etc.

Examples of the monomer having the imidazoyl group include 4-vinylimidazole, N-vinyl-2-ethylimidazole, N-vinyl-2-methylimidazole, etc.

Examples of the monomer having the pyridyl group include 2-vinylpyridine, 4-vinylpyridine, 2-methyl-5-vinylpyridine, etc.

The above-mentioned monomers may be used either singly or in combination of two or more of them.

Incidentally, the polymeric electrolyte having a negative charge and the polymeric electrolyte having a positive charge may have a building block derived from other monomer than the monomer having the negative charge or positive charge. Here, the other monomer is not specifically restricted, and known monomers can be used as the other monomer. Specific examples of the known monomers which can be used as the other monomer here include: salts such as sodium salts, potassium salts, ammonium salts, etc. of the above-mentioned monomers having the carboxyl group; monovalent metal salts, divalent metal salts, ammonium salts and organic amine salts of the above-mentioned monomers having the sulfo group; (poly)alkylene glycol di(meth)acrylates such as triethylene glycol di(meth)acrylate, (poly)ethylene glycol di(meth)acrylate, polypropylene glycol di(meth)acrylate, (poly)ethylene glycol(poly)propylene glycol di(meth)acrylate, etc.; bifunctional (meth)acrylates such as hexanediol di(meth)acrylate, trimethylolpropane tri(meth)acrylate, trimethylolpropane di(meth)acrylate, etc.; (poly)alkylene glycol dimalates such as triethylene glycol dimalate, polyethylene glycol dimalate, etc.; esters of an unsaturated monocarboxylic acid with an alcohol having 1 to 4 carbon atoms, such as methyl(meth)acrylate, ethyl(meth)acrylate, propyl(meth)acrylate, glycidyl(meth)acrylate, methyl crotonate, ethyl crotonate, propyl crotonate, etc.; amides of an unsaturated monocarboxylic acid with an amine having 1 to 30 carbon atoms, such as methyl(meth)acrylamide, etc.; vinyl aromatic compounds such as styrene, α-methylstyrene, vinyltoluene, p-methylstyrene, etc.; alkanediol mono(meth)acrylates such as 1,4-butanediol mono(meth)acrylate, 1,5-pentanediol mono(meth)acrylate, 1,6-hexanediol mono(meth)acrylate, etc.; dienes such as butadiene, isoprene, 2-methyl-1,3-butadiene, 2-chlor-1,3-butadiene, etc.; unsaturated amides such as (meth)acrylamide, (meth)acrylalkylamide, N-methyol(meth)acrylamide, N,N-dimethyl(meth)acrylamide, etc.; unsaturated nitriles such as (meth)acrylonitrile, α-chloroacrylonitrile, etc.; unsaturated esters such as vinyl acetate, vinyl propionate, etc.; and unsaturated amines such as aminoethyl(meth)acrylate, methylaminoethyl(meth)acrylate, dimethylaminoethyl(meth)acrylate, dimethylaminopropyl(meth)acrylate, dibutylaminoethyl(meth)acrylate, vinylpyridine, etc. The just-mentioned other monomers may be used either singly or in combination of two or more of them. Incidentally, in the case where the polymeric electrolyte further has the building block derived from the other monomer, the amount of the other monomer to be used is not specifically limited, insofar as it does not spoil the effect of the above-mentioned monomer having the positive charge or negative charge. The amount of the other monomer to be used is preferably 1 to 50 wt %, more preferably 1 to 10 wt %, based on the total amount of the monomers.

In (v) above, the electric charge of the polymeric electrolyte having a positive charge and the electric charge of the polymeric electrolyte having a negative charge are different in sign. In view of this, it suffices to select the above-mentioned monomers appropriately so that these polymeric electrolytes have respective charges of different signs, thereby producing the polymeric electrolytes having the desired charges. In this instance, the method for producing the polymeric electrolytes is not particularly restricted, and known polymerization methods can be used. Normally, it suffices for the above-mentioned monomers to be polymerized using a polymerization initiator. The method for polymerization of the monomer components is not specifically restricted; for example, the monomer components may be polymerized by polymerization in a solution, bulk polymerization, or the like. In the case where the polymeric electrolytes in the present invention are block copolymers or graft copolymers, nonlimitative examples of the polymerization method which can be used to produce the copolymers include living polymerization, polymerization using macromonomers, polymerization using a polymeric polymerization initiator, and polycondensation.

The above-mentioned hardening agent may be injected as it is into the respiratory region, but it may also be used in the state of being dissolved or dispersed in an appropriate solvent. The solvent which can be used in the latter case is not particularly restricted, insofar as it can dissolve or disperse the hardening agent and it is safe. Examples of the solvent which can be used include: water (exclusive of the case of (iii) above); dimethyl sulfoxide, dimethyl formamide; glycols such as ethylene glycol, diethylene glycol, propylene glycol, triethylene glycol, polyethylene glycol, polypropylene glycol, etc.; oils and fats such as olive oil, castor oil, squalane, lanolin, etc. The just-mentioned solvents may be used either singly or as mixture of two or more of them. Among these solvents, preferred are water, dimethyl sulfoxide, and dimethyl formamide, and more preferred is water. These are excellent in safety.

In the case where the hardening agent is injected into the respiratory region in the form of a solution or a dispersion, the concentration of the hardening agent in the solution or dispersion is not particularly limited. Preferably, the concentration is 1 to 10 wt %. Such a concentration ensures that the solution or dispersion has an appropriate degree of viscosity, so that the solution or dispersion forms a film on the inside walls of the alveolar parenchyma suffering from emphysema, easily and efficiently.

In (ii) to (v) above, the hardening agent is composed of a combination of two or more materials. The two or more materials may be injected through the same lumen of a catheter or may be injected through different lumens. Preferably, they are injected respectively through different lumens. This ensures that the two or more materials are mixed with one another in the alveolar parenchyma suffering from emphysema, so that the hardening reaction takes place assuredly in the alveolar parenchyma suffering from emphysema. In addition, the catheter is provided at its distal end with a plurality of lumen holes, and the area of contact between the catheter distal end and the respiratory region inside wall is small accordingly. Therefore, the distal end of the catheter would not easily adhered to the inside wall of the alveolar parenchyma. Besides, the injection timings for the two or more materials may be the same or different. Preferably, they are injected at the same time. This promises good mixing of the two or more materials, thereby permitting the hardening reaction to proceed efficiently and rapidly.

In addition, in using the hardening agent, a contrast medium opaque to X-rays or the like may further be used in combination. This ensures that the degree of filling of the alveolar parenchyma suffering from emphysema with the hardening agent can be confirmed under X-ray fluoroscopy. Consequently, the inner cavities of the alveolar parenchyma 3 suffering from emphysema can be filled up with the hardening agent 5 more assuredly and easily, while preventing the hardening agent from being loaded in an excessive amount to cause expansion of the alveolar parenchyma 3 suffering from emphysema. Here, the contrast medium is not specifically restricted insofar as it is opaque to radiations, and known radiopaque substances can be used. Specific examples of the contrast medium which can be used here include: iodine, barium, bismuth, boron, bromine, calcium, gold, platinum, silver, iron, manganese, nickel, gadolinium, dysprosium, tungsten, tantalum, stainless steel, Nitinol, and their compounds such as barium sulfate, etc. and their solutions/dispersions (e.g., physiological saline); amidotrizoic acid (3,5-diacetamino-2,4,6-triiodobenzoic acid), sodium meglumine amidotrizoate, meglumine amidotrizoate, sodium iotalamate, meglumine iotalamate, meglumine iotroxate, iotrolan, ioxaglic acid, ioxilan, iopamidol, iopromide, iohexyl, ioversol, iomeprol; and iodine addition products of ethylesters of the fatty acids obtained from poppy seed oil (e.g., Lipiodol™, which is poppy seed oil having carbon atom iodized), etc. These radiopaque substances may be used either singly or as mixture of two or more of them. In the case where the hardening agent has the contrast medium contained therein, the amount of the contrast medium mixed in is not particularly limited, insofar as it ensures that the filling of the alveolar parenchyma suffering from emphysema with the hardening agent can be confirmed under X-ray fluoroscopy.

The amount of the hardening agent, optionally with the contrast medium, injected (introduced) into the alveolar parenchyma suffering from emphysema is not particularly limited, insofar as it is sufficient for filling up the alveolar parenchyma suffering from emphysema with the hardening agent. For example, it suffices to inject the hardening agent, optionally with the contrast medium, while monitoring the injection pressure, and to stop the injection upon detection of a rise in the injection pressure. This ensures that the hardening agent fills up the alveolar parenchyma suffering from emphysema, to such an extent that gas exchange is substantially impossible.

Besides, the rate of injection (introduction) of the hardening agent, optionally with the contrast medium, into the alveolar parenchyma suffering from emphysema is not specifically restricted, insofar as it does not cause damage to the alveolar parenchyma suffering from emphysema. Preferably, the injection (introduction) rate is moderate. A moderate rate ensures that the inside of the alveolar parenchyma suffering from emphysema can be assuredly filled with the hardening agent, while preventing air from mixing in during the injection. Taking these points into account, the rate of injection (introduction) of the hardening agent, optionally with the contrast medium, into the alveolar parenchyma suffering from emphysema is preferably in the range of 30 to 2,000 mL/min, more preferably 300 to 1,200 mL/min. In the cases of (ii) to (v) above, the hardening agent is composed of two or more materials. In these cases, the injection (introduction) rates of the materials may be the same or different. Preferably, the injection (introduction) rates are substantially equal. This promises favorable mixing of the two or more materials with one another.

After the step (d) is finished, the catheter is pulled out. After the catheter is pulled out, the hardening agent is in the hardened state inside the alveolar parenchyma suffering from emphysema, as shown in FIGS. 1F and 2G, so that the alveolar parenchyma suffering from emphysema can be maintained in its shrunk form. Here, the catheter is preferably pulled out after the hardening agent has sufficiently been hardened inside the alveolar parenchyma suffering from emphysema. To be more specific, the catheter is preferably pulled out 1 to 10 minutes, more preferably 1 to 5 minutes, after the injection of the hardening agent. In this case, the balloon is preferably in its dilated state during when the catheter is kept indwelling in the respiratory region. This prevents the hardening agent from flowing out of the alveolar parenchyma suffering from emphysema, and permits a sufficient amount of the hardening agent to be hardened inside the alveolar parenchyma.

In this step (d), in order to prevent influences of pressure variations in the lung surrounding the objective region, it is desirable to once stop the lung ventilation and keep a constant pressure in the surroundings of the objective region. The pressure in this instance is desirably lower than the injection pressure in the catheter 1. For example, the surroundings can be held at a sustained positive pressure or an open atmospheric pressure. To be more specific, the bronchus or bronchiole on the central side relative to the objective bronchus or objective bronchiole is closed with the balloon, and, while keeping a constant pressure, the catheter 1 can be inserted into the objective bronchus or objective bronchiole. The part of the central-side bronchus to be closed may be the central trachea, but, preferably, the part is made to be the main bronchus or the peripheral side thereof; this permits ventilation for the remaining part to be continued. More specifically, the sheath 31 is disposed on the proximal side relative to the catheter 1 inserted in the alveolar parenchyma 3 suffering from emphysema. The balloon 31a disposes at the sheath 31 is put into closure, whereby the pressure (P1) in the bronchus or bronchiole between the balloons 1a and 31a (e.g., normal alveolar parenchyma) can be held at a pressure (P1<P2) lower than the injection pressure (P2) in the catheter 1, preferably at a sustained positive pressure or at an open atmospheric pressure. On the other hand, the balloon 1a disposed at the catheter 1 is put into closure, whereby the hardening agent 5 can be efficiently injected into the desired alveolar parenchyma 3 suffering from emphysema, while preventing the hardening agent 5 from flowing back to the trachea (proximal) side of the bronchus or bronchiole 2. Here, the method for controlling the pressure (P1) in the bronchus and bronchiole between the balloons 1a and 31a (e.g., normal alveolar parenchyma) and the injection pressure (P2) in the catheter 1 is not specifically restricted. Preferably, as shown in FIG. 3B, a sealing stopcock 32 is provided on the proximal side of the sheath 31, and the catheter 1 is inserted into the sheath 31 via the sealing stopcock 32. With the sealing stopcock 32 thus provided, the inside of the alveolar parenchyma on the distal (peripheral) side relative to the sheath 31 can be made to be a closed system, so that control of pressure in this part can be easily carried out. In addition, where a three-way stopcock 33 is provided at a proximal portion of the sheath 31 and a gas 38 is introduced or sucked through the three-way stopcock 33, the pressure (P1) in the bronchus or bronchiole between the balloons 1a and 31a (e.g., normal alveolar parenchyma) is appropriately controlled, preferably to a sustained positive pressure or an open atmospheric pressure. In addition, the injection pressure in the catheter 1 can also be controlled in the same manner. Specifically, as shown in FIG. 3B, a sealing stopcock 35 is provided on the proximal side of the catheter 1. With the sealing stopcock 35 thus provided, the inside of the alveolar parenchyma on the distal (peripheral) side relative to the catheter 1 can be made to be a closed system, so that the control of pressure in this part can be easily performed. This permits selective filling with a gas or gasses to be carried out easily. In addition, where a three-way stopcock 36 is provided on the proximal side of the catheter 1 and a gas 39 is introduced or sucked through the three-way stopcock 36, the injection pressure (P2) in the catheter 1 can be controlled appropriately.

As above-mentioned, according to the method pertaining to the present invention, air stagnant in the alveolar parenchyma suffering from emphysema can be efficiently removed, and the thus reduced volume can be maintained during respiration. This makes it possible to suppress or restrain overexpansion of the lung, which is one of the causes of weakening of the patient due to emphysema or occlusion of the bronchi. Besides, the alveolar parenchyma suffering from emphysema can be restored to its original size or less, whereby compression or occlusion of the surrounding bronchi by the surrounding alveolar parenchyma can be suppressed or prevented. Further, the method for treatment pertaining to the present invention resides in therapy by way of a catheter and does not need a surgical treatment, whereby the burden on the patient can be reduced.

### EXAMPLES

Now, a method for coating the tissue of alveolar sacs (air sacs) or pulmonary alveoli, the tissue of a terminal bronchiole, and the tissue of a bypass respiratory tract at an arbitrary part in a per-bronchial manner will be described in detail below, based on preferred embodiments. It should be noted here, however, that the technical scope of the present invention is not to be limited only to the following examples.

### Example 1

As shown in FIG. 1A, an OTW-type PTCA balloon catheter 1 [Ryujin Plus OTW (registered trademark); Medical Instrument Approval Number: 21600BZZ00035; made by TERUMO CORPORATION] for use in treatment of stenosis of blood vessel lumen in a cardiovascular region was inserted through a working lumen (not shown) of a bronchoscope into the lumen of a bronchiole 2. Here, a guide wire [Runthrough (registered trademark), made by TERUMO CORPORATION] (outside diameter: 0.014 inch) was preliminarily inserted into the working lumen of the bronchoscope. The distal end of the guide wire was advanced into the vicinity of a desired alveolar parenchyma 3 suffering from emphysema under X-ray fluoroscopy. Next, a catheter was advanced through the function of the guide wire into the vicinity to the desired alveolar parenchyma 3 suffering from emphysema under X-ray fluoroscopy, followed by pulling out the guide wire.

Subsequently, as shown in FIG. 1B, using a syringe connected to a balloon part dilating lumen arranged at a proximal portion of the catheter 1, the balloon 1a was dilated with air, to close the bronchiole 2.

A 30 mL syringe was connected via a three-way stopcock to a shrinking lumen arranged at a proximal portion of the catheter 1. By pulling this syringe, as shown in FIG. 1C, a residual gas 4 in the alveolar parenchyma 3 suffering from emphysema was removed by suction, whereby the alveolar parenchyma 3 suffering from emphysema was shrunk. By this operation, the bronchiole 2 on the peripheral side relative to the balloon part is collapsed, and the inside surface of the bronchus is pressed against the surface of the balloon part, so that the inside of the alveolar parenchyma 3 suffering from emphysema is kept in a negative-pressure state.

Another 30 ml syringe was filled with octyl α-cyanoacrylate prepared as a film-forming agent 5. While the inside of the alveolar parenchyma 3 suffering from emphysema was kept at the negative pressure, the three-way stopcock disposed between the above-mentioned syringe and the catheter was closed, and the syringe filled with octyl α-cyanoacrylate was connected to this three-way stopcock.

Next, as shown in FIG. 1D, the above-mentioned three-way stopcock was opened, octyl α-cyanoacrylate was injected from the syringe into the inner cavities of the alveolar parenchyma 3 suffering from emphysema through the lumen of the catheter 1. Upon a rise in the injection pressure of the syringe, the injection of octyl α-cyanoacrylate was stopped. As a result, a sufficient amount of octyl α-cyanoacrylate was made to fill up the inner cavities of the alveolar parenchyma 3, and reacted with water present on the surfaces of the alveolar parenchyma 3 suffering from emphysema, thereby starting to be hardened (FIG. 1E). After the injection of octyl α-cyanoacrylate, the system was left to stand for three minutes, thereby allowing the octyl α-cyanoacrylate in the alveolar parenchyma 3 suffering from emphysema to be hardened sufficiently.

Further, the balloon 1a was shrunk, and the condition of the lung was observed through X-ray imaging. After it was confirmed that the alveolar parenchyma 3 in the part filled with the octyl α-cyanoacrylate injected was not enlarged by inflow of air, the catheter 1 was pulled out (FIG. 1F). It was confirmed that the volume of the alveolar parenchyma 3 suffering from emphysema can be reduced by the process of this example and that the alveolar parenchyma 3 suffering from emphysema is held in the reduced-volume state.

### Example 2

As shown in FIG. 2A, a microcatheter (for example, FINECROSS (registered trademark), made by TERUMO CORPORATION) 1 was inserted through a working lumen (not shown) of a bronchoscope into the lumen of a bronchiole 2. Here, a guide wire [Runthrough (registered trademark), made by TERUMO CORPORATION] (outside diameter: 0.014 inch) was preliminarily inserted in the working lumen of the bronchoscope. The distal end of the guide wire was advanced into the vicinity of a desired alveolar parenchyma 3 suffering from emphysema under X-ray fluoroscopy. Next, a catheter was advanced through the function of the guide wire into the vicinity of the desired alveolar parenchyma 3 suffering from emphysema under X-ray fluoroscopy, followed by pulling out the guide wire.

Subsequently, as shown in FIG. 2B, using an indeflator connected to a balloon part dilating lumen disposed at a proximal portion of the catheter 1, a balloon 1a was dilated with air, thereby closing a bronchiole 2. As shown in FIG. 2C, a porous ceramic powder as a gas-absorbing agent 6 was sprayed through a lumen through which gas can be fed of the catheter 1 into the inner cavities of the alveolar parenchyma 3 suffering from emphysema. The porous ceramic powder thus sprayed absorbed a gas remaining in the alveolar parenchyma suffering from emphysema; as a result, the alveolar parenchyma 3 suffering from emphysema was shrunk, and the volume thereof was reduced (FIG. 2D).

As shown in FIG. 2E, another 30 ml syringe was filled with octyl α-cyanoacrylate prepared as a film-forming agent 5. The octyl α-cyanoacrylate was injected from the syringe into the inner cavities of the alveolar parenchyma 3 suffering emphysema, through the lumen of the catheter 1. In this instance, by pulling a syringe connected to another negative-pressure lumen of the catheter 1, a residual gas 4 in the alveolar parenchyma 3 suffering from emphysema was removed by suction, and the inside of the alveolar parenchyma 3 suffering from emphysema was kept at a negative pressure (FIG. 2E). Upon a rise in the injection pressure of the syringe, the injection of octyl α-cyanoacrylate was stopped. As a result, a sufficient amount of octyl α-cyanoacrylate was made to fill up the inner cavities of the alveolar parenchyma 3, and reacted with water present on the surfaces of the alveolar parenchyma 3 suffering from emphysema, thereby starting to be hardened rapidly.

Here, simultaneously with the start of the injection of octyl α-cyanoacrylate, a waterdrop was dropped onto octyl α-cyanoacrylate preliminarily dropped onto a slide glass, and the state of reaction (hardening) of the octyl α-cyanoacrylate with water was thereby observed.

After it was confirmed that the octyl α-cyanoacrylate was hardened sufficiently (FIG. 2F), the balloon 1a was shrunk. The condition of the lung was observed under X-ray imaging. After it was confirmed that the alveolar parenchyma 3 was not enlarged by inflow of air, the catheter 1 was pulled out. It was confirmed that the volume of the alveolar parenchyma 3 suffering from emphysema can be reduced by the process of this example and that the alveolar parenchyma 3 suffering from emphysema is held in the reduced-volume state.

1. A method for treatment of emphysema comprising:
(a) inserting a catheter having a balloon into a bronchus or bronchiole;
(b) dilating the balloon to occlude the bronchus or bronchiole;
(c) shrinking pulmonary alveoli or alveolar sacs on a downstream side of the bronchus or bronchiole occluded by the step (b); and
(d) injecting a hardening agent through the catheter into the respiratory region and hardening the hardening agent.

2. The method for treatment of emphysema according to claim 1,
wherein in the step (c), the shrinking of the pulmonary alveoli or alveolar sacs is carried out by:
(c-1) removing a gas remaining in the respiratory region inclusive of the pulmonary alveoli or alveolar sacs by suction through the catheter; or
(c-2) injecting a gas-absorbing agent for absorption of air present in the pulmonary alveoli or alveolar sacs into the pulmonary alveoli or alveolar sacs.

3. The method for treatment of emphysema according to claim 1, wherein in the step (d), the hardening agent is injected through the catheter into the respiratory region, while sucking the gas remaining in the pulmonary alveoli or alveolar sacs through the catheter and thereby keeping the inside of the respiratory region at a negative pressure.

4. The method for treatment of emphysema according to claim 2, wherein in the step (d), the hardening agent is injected through the catheter into the respiratory region, while sucking the gas remaining in the pulmonary alveoli or alveolar sacs through the catheter and thereby keeping the inside of the respiratory region at a negative pressure.
